(19) United States
(12) Patent Application Publication
(10) Pub. No.: US 2009/0143683 A1
(43) Pub. Date: Jun. 4, 2009

(54) DIAGNOSTIC ULTRASOUND APPARATUS

(75) Inventors: Haitao HUANG, Shenzhen (CN); Xujin HE, Shenzhen (CN); Zhiyong GUAN, Shenzhen (CN); Guotao LIU, Shenzhen (CN)

Correspondence Address: MINDRAY C/O STOEL RIVES LLP, 201 S. MAIN STREET, SUITE 1100, SALT LAKE CITY, UT, 84111, US

(73) Assignee: SHENZHEN MINDRAY BIO-MEDICAL ELECTRONICS CO., LTD., Shenzhen (CN)

(21) Appl. No.: 12/270,610

(22) Filed: Nov. 13, 2008

(30) Foreign Application Priority Data
Nov. 30, 2007 (CN) 200710077460.4

(51) Int. Cl. A61B 8/14

(52) U.S. Cl. 600/459

(57) ABSTRACT

A diagnostic ultrasound apparatus is disclosed comprising a base board, an ultrasonic front-end analog circuit, a digital processing circuit connected with the ultrasonic front-end analog circuit, and a power module for providing electric power to the ultrasonic front-end analog circuit and the digital processing circuit. The ultrasonic front-end analog circuit is implemented with a modular design, the digital processing circuit and the power module being located at one half-region of the base board, and the ultrasonic front-end analog circuit being located at the other half-region of the base board. The ultrasonic front-end analog circuit is provided far away from the power module and the digital processing circuit such that interference from the power supply and digital circuits is prevented.

### CROSS-REFERENCE TO RELATED APPLICATIONS

This application claims priority to Chinese Patent Application No. 200710077460.4, filed Nov. 30, 2007, for “Diagnostic Ultrasound Apparatus,” which is incorporated herein by reference in its entirety.

### TECHNICAL FIELD

The present disclosure relates to a portable diagnostic ultrasound apparatus.

### BRIEF SUMMARY

A diagnostic ultrasound apparatus includes a base board, an ultrasonic front-end analog circuit, a digital processing circuit connected with the ultrasonic front-end analog circuit, and a power module for providing electric power to the ultrasonic front-end analog circuit and the digital processing circuit. The ultrasonic front-end analog circuit may be implemented with a modular design, in which the digital processing circuit and the power module are located at one half-region of the base board, and the ultrasonic front-end analog circuit is located at the other half-region of the base board.

### BRIEF DESCRIPTION OF THE DRAWINGS

FIG. 1 is a circuit schematic diagram of a conventional diagnostic ultrasound apparatus;

FIG. 2 shows a layout of a hardware circuit according to an embodiment of the present disclosure; and

FIG. 3 shows a detailed layout of the hardware circuit according to an embodiment of the present disclosure.

### DETAILED DESCRIPTION

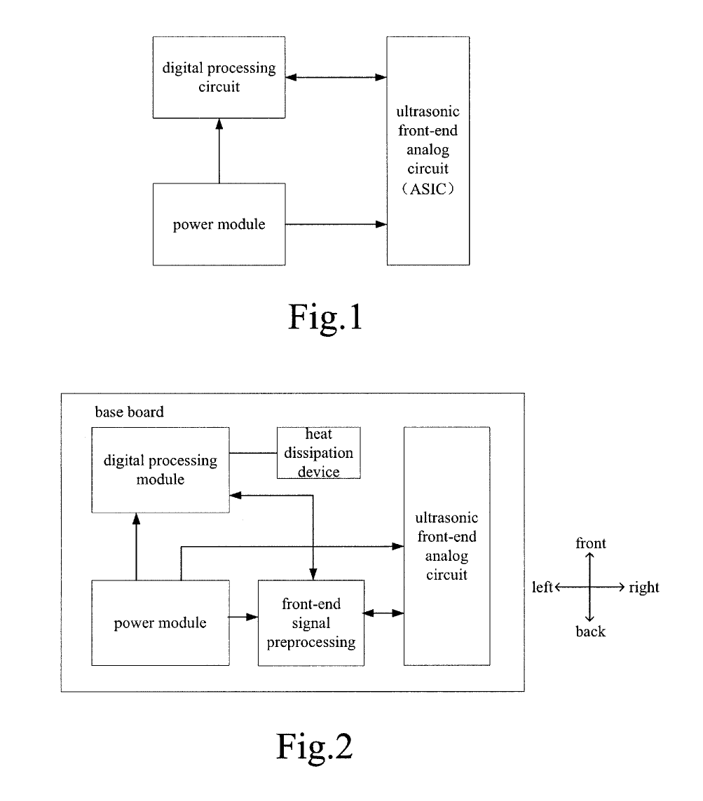

As shown in FIG. 1, a hardware circuit of a portable diagnostic ultrasound apparatus generally comprises an ultrasonic front-end analog circuit, a digital processing circuit (e.g., a computer processing module) for processing and controlling echo signals from the ultrasonic front-end analog circuit, and a power module for providing electric power to the ultrasonic front-end analog circuit and the digital processing circuit. The ultrasonic front-end analog circuit comprises a transmitting part and a receiving part, wherein ultrasonic signals are transmitted generally at high voltage by the transmitting part and low-noise echo signals are required at the receiving part. As a result, a typical ultrasonic front-end analog circuit requires very high performance characteristics.

In existing systems, the ultrasonic front-end analog circuit is made in the form of a ASIC chip packaged using BGA. However, once the ultrasonic front-end analog circuit is formed as an ASIC chip, it is troublesome to disassemble and recover the ASIC chip due to its high integration level when the image quality is determined to be degraded for the performance of a certain channel after completing the assembly as a whole. Furthermore, since the number of internal channels has already been determined during the ASIC design, it is generally not possible to change the number of internal channels in the diagnostic ultrasound apparatus in accordance with particular demands.

A aspect of the present disclosure is a diagnostic ultrasound apparatus in which an ultrasonic front-end analog circuit is implemented with a standalone modular design, such that the ultrasonic front-end analog circuit can have a satisfactory anti-interference performance.

In one embodiment, the diagnostic ultrasound apparatus comprising a base board, an ultrasonic front-end analog circuit, a digital processing circuit connected with the ultrasonic front-end analog circuit, and a power module for providing electric power to the ultrasonic front-end analog circuit and the digital processing circuit. The ultrasonic front-end analog circuit may be implemented with a modular design, such that the digital processing circuit and the power module are located at one half-region of the base board and the ultrasonic front-end analog circuit is located at the other half-region of the base board. In one embodiment, the power module and a channel selecting module of the ultrasonic front-end analog circuit are diagonally arranged on the base board.

The disclosed portable diagnostic ultrasound apparatus of the present disclosure utilizes a modular ultrasonic front-end analog circuit, and various modules and channels are designed independently so that it is convenient to assemble, maintain, and subsequently upgrade the diagnostic ultrasound apparatus. Furthermore, according to the present disclosure, the ultrasonic front-end analog circuit is provided sufficiently far away from the power module and the digital processing circuit so as to prevent interference from the power supply and digital circuits and enable the ultrasonic front-end analog circuit to meet anti-interference requirements.

Referring to FIG. 2, the hardware circuit of a portable diagnostic ultrasound apparatus may comprise a base board, an ultrasonic front-end analog circuit, a digital processing circuit, and a power module. The ultrasonic front-end analog circuit transmits ultrasonic waves, receives ultrasonic echo signals, and performs a amplification and a analog-digital conversion on the echo signals. The digital processing circuit implements loading of an operating system, system control, post-processing of images, user application interfaces, and external interfaces. The power module provides electric power to the ultrasonic front-end analog circuit and the digital processing circuit. The base board provides interconnections among a plurality of functional modules, as well as other standard functions.

In one embodiment, the power module is located on the left half-region of the base board with respect to the user and close to the user i.e., a proximal left half-region of the base board. The power module is separated from the other modules and is well shielded. A board to board connection may be made between the power module and the base board in the form of a plug-socket mechanism.

The digital processing circuit may be located on the left half-region of the base board with respect to the user, far away from the user i.e., a distal left half-region of the base board and mounted on the base board in the form of a daughter board. Since sufficient space between the base board (the carrier board) and the daughter board is provided, a part of the peripheral circuits of the digital processing circuit may still be arranged on the base board area directly under the daughter board to make full use of the compact space therebetween.

In one embodiment, the ultrasonic front-end analog circuit is located on the right half-region of the base board with respect to the user. Considering the high power density of the digital processing circuit, a heat dissipation device for heat radiation of the digital processing circuit is provided between the digital processing circuit and the ultrasonic front-end analog circuit. The dedicated heat dissipation device may be omitted if power consumption of the digital processing circuit is low. A front-end signal preprocessing circuit is provided between the power module and the ultrasonic front-end analog circuit. The front-end signal preprocessing circuit is electrically connected with the digital processing circuit and the ultrasonic front-end analog circuit, respectively.

Thus, the ultrasonic front-end analog circuit is located far away from the power module and the digital processing circuit so as to avoid interference therefrom.

In one embodiment, a shielding case for shielding electromagnetic radiation is provided around the ultrasonic front-end analog circuit to avoid interference from the power module and the digital processing circuit.

The separation of the ultrasonic front-end analog circuit from the power module and the digital processing circuit effectively prevents strong EMC radiation transmitted from the power module and the digital processing circuit, thereby ensuring compliance with EMC regulation.

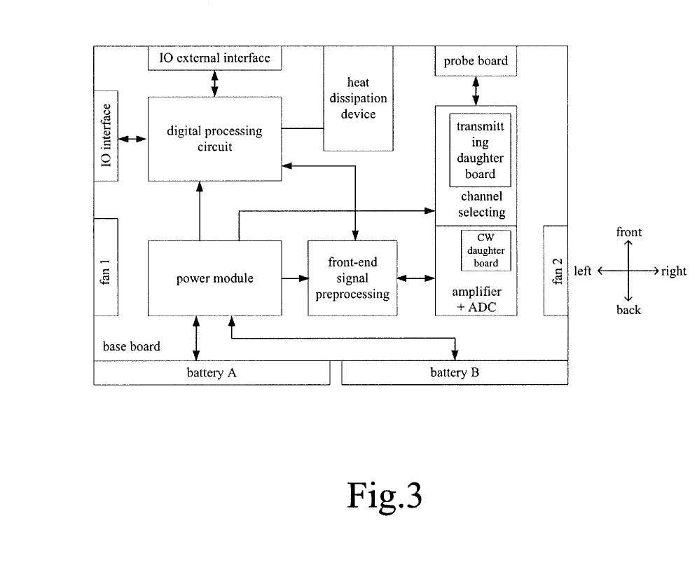

As shown in FIG. 3, the ultrasonic front-end analog circuit comprises a channel selecting module, a transmitting module, a CW (continuous wave Doppler) module, an amplifier, and an analog-digital conversion module. The channel selecting module is a circuit for selecting desired array elements in a probe for ultrasonic imaging. The transmitting module is a circuit for transmitting ultrasonic waves. The CW module is a circuit for implementing ultrasonic continuous wave Doppler imaging. The amplifier and the analog-digital conversion module are circuits for performing amplification and analog-digital conversion on echoes from the probe.

In one embodiment, the channel selecting module and the transmitting module are located on the right half-region of the base board with respect to the user and far away from the user, i.e., a distal right half-region of the base board. The amplifier and the analog-digital conversion module are located on the right half-region of the base board with respect to the user and close to the user, i.e., a proximal right half-region of the base board. The CW module may be located above the amplifier and the analog-digital conversion module in the form of a daughter board. A probe board electrically connected to the channel selecting module may be provided at the most forward part of the base board with respect to the user, i.e., at the forward part of the channel selecting module. The probe board may have a board to board connection to the base board in the form of a plug-socket mechanism. The transmitting module may be connected with the base board in the form of daughter board and located above the channel selecting module.

In one embodiment, a second fan 2 for heat dissipation of the amplifier and the analog-digital conversion circuit is provided at an edge portion of the base board (e.g., the right edge portion of the base board with respect to the user) close to the amplifier and the analog-digital conversion module of the ultrasonic front-end analog circuit.

Batteries electrically connected with the power module, such as batteries A and B shown in FIG. 3, are provided at an edge portion of the base board close to the power module (e.g., the edge portion of the base board nearby the user). The batteries A and B are identical and exchangeable with each other. Alternatively, only one battery may be used as needed.

A first fan 1 for heat dissipation of the power module is provided at another edge portion of the base board nearby the power module (e.g., the left edge portion of the base board with respect to the user).

Interfaces electrically connected with the digital processing circuit are provided at an edge portion of the base board close to the digital processing circuit. IO interfaces available to the user that are arranged on the left of the digital processing circuit may include a USB interface, a network interface, a video signal interface (e.g., S-VIDEO), and a power adapter interface, etc. IO extension interfaces are arranged in front of the digital processing circuit (i.e., far away from the user). By using special external IO extenders, these IO extension interfaces may be extended as user IO interfaces, such as USB interfaces, network interfaces, S-VIDEO interfaces, parallel interfaces, and VGA interfaces.

The layout method will be described in four aspects as follows:

1. Performance

Such the layout as the power module being located at the left-rear part of the diagnostic ultrasound apparatus, the digital processing circuit being located at the left-front part of the diagnostic ultrasound apparatus, the ultrasonic transmitting and receiving channel selecting circuit being located at the right-front part of the diagnostic ultrasound apparatus and the amplifier and analog-digital conversion module being located at the right-rear part of the diagnostic ultrasound apparatus ensures that the front-end analog circuit with weak signals is separated and far away from the power module and the digital processing circuit, which effectively prevents interference from the power supply and digital circuits. The shielding case of the analog circuit further improves anti-interference capability.

2. Function

Such the layout as the digital processing circuit being located at the left-front part of the diagnostic ultrasound apparatus, user IO interfaces and IO extension interfaces being respectively arranged on the left and front of the digital processing circuit effectively meets the shaping and user requirements, minimizes the lengths of PCB traces between the user IO interfaces and the digital processing circuit, and ensures performance of user interfaces.

3. Heat Dissipation

Because the power density of the digital processing circuit is high, a heat dissipation device composed of fins and fan is provided at the right side of the digital processing circuit. A special heat dissipation block is provided above the digital processing circuit and a number of heat pipes are provided therein to transfer heat generated from the digital processing circuit to the fins on the right side and the heat is transferred into the atmosphere by the fan.

The power module also generates a large amount of heat, and thus a fan 1 is provided at the left of the power module to improve ventilation and heat dissipation.

Because a large amount of heat is generated from the amplifier and the analog-digital conversion module (ADC) of the analog circuit, a fan 2 is provided at the right of the amplifier and ADC to improve ventilation and heat dissipation.

4. EMC

Separating the ultrasonic front-end analog circuit, the power module, and the digital processing circuit effectively prevents strong EMC radiation transmitted from the power module and the digital processing circuit, thereby ensuring compliance with EMC regulation.

In the above-mentioned embodiment, the position of the ultrasonic front-end analog circuit with respect to the power module and the digital processing circuit may also be changed by rotation or reversion. For example, the power module and the digital processing circuit may be located at the right side of the base board and the ultrasonic front-end analog circuit may be located at the left side of the base board.

Methods of connections among various modules are not limited to such means as a daughter board or board to board connection with a plug-socket mechanism. Other means (such as cable connection) may also be used to realize connections between these modules.

The daughter board is not limited to a board with one layer, but could include two or more layers. As for the transmitting module and the CW module, it may not necessary to use the daughter board, and they may also be implemented directly on the base board.

The channel selecting circuit, the amplifier, and the analog-digital conversion module are not limited to being arranged on the base board. The entire analog circuit (including the transmitting module and CW module) may be separately made into a module formed by one PCB or a module comprised of several PCBs connected by daughter-carrier boards. This analog circuit module may be connected to the base board by means of a cable connection or a board to board connection with a plug-socket mechanism.

The functions of the digital processing circuit may be realized by an IPC (industry personal computer) module, an embedded system, a FPGA, a DSP and an ASIC, and may be directly implemented on the base board rather than by daughter board.

The above detailed description should be understood as an illustration of specific embodiments of the present invention, and not as a limitation of the invention. It will be apparent to those skilled in the art that variations, alternatives, and alterations may be made to the embodiments herein disclosed without departing from the spirit or scope of the present invention.

What is claimed is:

1. A diagnostic ultrasound apparatus, comprising:
a base board,
an ultrasonic front-end analog circuit,
a digital processing circuit connected with the ultrasonic front-end analog circuit, and
a power module for providing electric power to the ultrasonic front-end analog circuit and the digital processing circuit,
wherein the ultrasonic front-end analog circuit is implemented with a modular design, and
wherein the digital processing circuit and the power module are located at one half-region of the base board, and the ultrasonic front-end analog circuit is located at the other half-region of the base board.

2. The diagnostic ultrasound apparatus of claim 1, wherein the power module and a channel selecting module of the ultrasonic front-end analog circuit are diagonally arranged on the base board.

3. The diagnostic ultrasound apparatus of claim 2, wherein a heat dissipation device for providing heat dissipation processing for the digital processing circuit is provided between the digital processing circuit and the ultrasonic front-end analog circuit.

4. The diagnostic ultrasound apparatus of claim 3, wherein a front-end signal preprocessing circuit is provided between the power module and the ultrasonic front-end analog circuit, and the front-end signal preprocessing circuit is electrically connected with the digital processing circuit and the ultrasonic front-end analog circuit, respectively.

5. The diagnostic ultrasound apparatus of claim 2, wherein the base board comprises a proximal left half-region, a distal left half-region, a proximal right half-region, and a distal right-half region, wherein the power module is located at the proximal left half-region of the base board, wherein the digital processing circuit is located at the distal left half-region of the base board, wherein the channel selecting module and a transmitting module of the ultrasonic front-end analog circuit are located at the distal right half-region of the base board, and wherein an amplifier and an analog-digital conversion module of the ultrasonic front-end analog circuit are located at the proximal right half-region of the base board.

6. The diagnostic ultrasound apparatus of claim 5, wherein the power module is connected with the base board in the form of a plug-socket mechanism, the digital processing circuit is connected with the base board in the form of a daughter board and the transmitting module is connected with the base board in the form of a daughter board and located above the channel selecting module.

7. The diagnostic ultrasound apparatus of claim 1, wherein a shielding case for shielding radiated interference from the power module and the digital processing circuit is provided at the periphery of the ultrasonic front-end analog circuit.

8. The diagnostic ultrasound apparatus of claim 7, wherein a battery electrically connected with the power module and a first fan for heat dissipation of the power module are provided at an edge of the base board close to the power module.

9. The diagnostic ultrasound apparatus of claim 7, wherein an external user interface electrically connected with the digital processing circuit is provided at an edge of the base board close to the digital processing circuit.

10. The diagnostic ultrasound apparatus of claim 7, wherein a second fan for heat dissipation of the ultrasonic front-end analog circuit is provided at an edge of the base board close to the amplifier and the analog-digital conversion module of the ultrasonic front-end analog circuit.
